(19) United States
(12) Patent Application Publication
(10) Pub. No.: US 2007/0048343 A1
(43) Pub. Date: Mar. 1, 2007

(54) Biocidal premixtures

(75) Inventors: Natalie A. Merrill, Somerville, NJ (US); James E. Garft, Yardley, PA (US); John D. Clay, Randolph, NJ (US)

Correspondence Address: COLLEEN D. SZUCH;Honeywell International Inc. - Patent Department, 101 Columbia Road, Morristown, NJ, 07962, US

(73) Assignee: Honeywell International Inc.

(21) Appl. No.: 11/212,341

(22) Filed: Aug. 26, 2005

(51) Int. Cl. A61K 31/695; B32B 21/02; A01N 25/00; A01N 55/00

(52) U.S. Cl. 424/405; 514/63; 428/292.4

(57) ABSTRACT

This invention provides a novel biocidal premixture, particularly useful as a component in cellulose reinforced thermoplastic composites. The inventive biocidal premixture includes a combination of a biocidal agent, either a coupling agent, a lubricant, or both. The addition of such biocidal premixtures to cellulose reinforced thermoplastic composite materials offers improved resistance to attack by molds, fungus, algae, and the like, improved adhesion stability, greater moisture resistance and aging resistance, limited leaching, enhanced toughness moduli, and increased efficiency in such composites. Products using the inventive biocidal premixtures are useful in forming materials for exterior building applications such as decks, siding, roofing, windows, moldings, docks, wharfs and the like.

### BACKGROUND OF THE INVENTION

1. Field of the Invention

The present invention relates to biocidal premixtures useful as components of composite materials. These biocidal premixtures are particularly useful as components of cellulose-containing composites which include a cellulosic material and a thermoplastic polymer. Such cellulose-containing composites, or wood-plastic composites, have many uses in various industrial and structural applications.

2. Description of the Related Art

Wood plastic composite materials have been long known in the art. Typically these materials include a cellulosic material, an organic polymer, and a filler. The composite materials are cast into various shapes to form materials having the look and feel of natural wood, and are used in the formation of outdoor materials including decks, siding, roofing, windows, moldings, docks, wharfs and the like.

Unfortunately, the wood-plastic composite industry is challenged by issues involving decomposition due to biological attack. Algae, mold, fungus, mildew, and the like, attack these materials and affect the physical properties, aging resistance, structural integrity, and appearance of cellulose reinforced thermoplastic composites. A number of anti-biologic additives, such as biocides and algicides, have been designed for use as a component of such composite materials in an attempt to negate these problems. However, conventional anti-biologic additives (i.e. zinc borate) used in these applications have had limited efficacy.

Another known problem in the industry is the leaching of metal-based biocides and algicides used in wood applications. The leaching of these materials can pose a significant risk to water supplies, aquatic wildlife and benthic communities, causing further risk to other organisms up the food chain. Thus, the need exists for a biocidal component for cellulose reinforced thermoplastic composites, which biocidal component delays mold, mildew, microbial and biological attack or degradation, while providing aging stability as well as limited environmental leaching.

Another problem associated with the formation of wood-plastic composites is the difficulty in processing and binding cellulosic materials with polymeric materials. Thus, the need exists for a biocidal material which also facilitates processing and improves binding and adhesion stability between cellulose and polymeric materials in the formation of such composites. Still another drawback of wood based composites is their susceptibility to moisture that leads to softening and material degradation. Thus, there is a need for water resistance to an article made from wood composite.

The present invention offers a solution to these problems. This invention provides a novel biocidal premixture for use as a component of cellulose reinforced thermoplastic composites. The inventive biocidal premixture includes a combination of a biocidal agent and either a coupling agent, a lubricant, or both. The biocidal agent inhibits biological attack on composite materials, the coupling agent offers enhanced adhesion stability, and the lubricant serves as a release agent and/or processing aid.

The addition of the inventive biocidal premixture to a cellulose reinforced thermoplastic composite offers a significant improvement over the prior art, including but not limited to improved resistance to biologic attack by molds, fungus, algae, and the like, improved adhesion stability, greater moisture resistance and aging resistance, improved strength, better extrudability, limited leaching, enhanced toughness moduli, and increased efficiency in such composites. Products using the inventive biocidal premixture are particularly useful in forming materials for exterior building applications, including but not limited to decks, siding, roofing, windows, moldings, docks, wharfs and the like.

### SUMMARY OF THE INVENTION

The invention provides a biocidal premixture comprising a combination of a biocidal agent and at least one or both of:
a) a coupling agent; and/or
b) a lubricant.

The invention further provides a cellulosic composition which comprises:
a) a cellulosic material;
b) a thermoplastic polymer; and
c) the biocidal premixture comprising a combination of a biocidal agent and at least one or both of:
i) a coupling agent; and/or
ii) a lubricant.

The invention further provides a cellulosic composition which comprises:
a) a cellulosic material;
b) a thermoplastic polymer; and
c) the biocidal premixture comprising a combination of a biocidal agent and at least one or both of:
i) a coupling agent; and/or
ii) a lubricant.

The invention still further provides a process for forming a cellulosic composition which comprises:
a) first forming a biocidal premixture comprising a combination of a biocidal agent and at least one or both of:
i) a coupling agent; and/or
ii) a lubricant; and
b) thereafter combining the biocidal premixture with a cellulosic material and a thermoplastic polymer to thereby form a cellulosic composition.

The invention further provides a biocidal compound comprising a biocidal agent nucleophilically grafted to a coupling agent.

### DETAILED DESCRIPTION OF THE INVENTION

The invention relates to a biocidal premixture for use in various applications, such as exterior material applications. The inventive biocidal premixture is useful as a component of composite compositions, and particularly when combined with a cellulosic material and a polymeric material in the formation of a cellulosic composition for wood-plastic composites and the like.

The biocidal premixture comprises a combination of a biocidal agent and at least one coupling agent, at least one lubricant, or both. In a preferred embodiment of the invention, the biocidal premixture includes a biocidal agent and coupling agent without a lubricant, another embodiment includes a biocidal agent and lubricant without a coupling agent, and still another embodiment includes a biocidal agent with both a coupling agent and a lubricant.

An important feature of the present invention is that the biocidal agent and the coupling agent and/or lubricant are first combined with each other to form the biocidal premixture, followed by a subsequent combining of the premixture with other components of a composite composition. This order of combination provides several advantages, including an enhanced lifetime of the composite and its components. The order of combination not only provides a uniform, even combining of the biocidal premixture's components within the premixture itself, but also allows a uniform, even combining of the biocidal premixture within the overall composite composition. The order of combination also affords better efficiency of the biocidal agent, by positioning the biocidal agent in better proximity to composite surfaces which are in need of the biocidal agent's effect.

The biocidal agent may comprise any anti-biologic chemical moiety which is capable of halting, limiting, inhibiting, or preventing the growth, formation, attraction, or spreading of undesirable biological growths and/or organisms such as mold, mildew, algae, microbes, fungi, bacteria, mites, insects, and the like. Materials suitable for use as a biocidal agent nonexclusively include algicides, herbicides, antifungals, antimicrobials, antibacterials, antibiotics, termiticides, miticides, insecticides, and combinations thereof. Some examples of suitable conventional biocidal agents nonexclusively include: algicides including EPA-registered algicides, such as chelated copper, elemental copper, and copper sulfate pentahydrate; herbicides including EPA-registered herbicides, such as diquat dibromide, fluridone; anti-biologics including EPA registered anti-biologics, commercially produced biocides including quaternary ammonium salts, metal-based systems and anti-microbial systems for use in polyolefins; antifungals or fungicides such as copper sulphate, chromated copper arsenate, ammoniacal copper zinc arsenate, alkylammonium compounds such as didecylmethylammonium chloride, ammoniacal copper-alkylammonium compounds such as ammoniacal copper-didecylmethylammonium chloride, and combinations thereof; herbicides such as the triazine herbicides atrazine, simazine, duiron, terbuthylazine, alachlor, bromacil, metolachlor, and/or precursors of these materials such as 2,4,6, trichloro-1,3,5 triazine, and combinations thereof; copper 8-quinolinolate; substituted isothiazolones such as 4,5-dichloro-2-n-octyl-4-isothiazolin-3-one; 2,4,5,6-tetrachloroisophthalonitrile; a thiazole such as 2-thiocyanomethylthiobenzothiazole (TCMTB); methylene-bis-thiocyanate (MBT); a carbamate such as 3-iodo-propenyl butyl carbamate; triazoles such as azaconazole, propiconazole, and tebuconazole; and termiticides such as chlorpyriphos, silafluofen, pyrethroids, copper 8 quinolate, zinc naphthanate, and copper naphthanate; and combinations thereof.

Preferred biocidal agents comprise non-leaching, emulsifiable materials. By non-leaching it is meant that the biocidal agent substantially retains its anti-biologic characteristics, and resists leaching, leaking, dissolving, evaporation, dissipation, or other loss of all or part of its active anti-biologic ingredient into the environment over time. A non-limiting example of suitable non-leaching biocides includes polyhedral oligomeric silsesquioxane (hereinafter referred to as “POSS”) moieties. Examples of suitable POSS moieties nonexclusively include octafunctional quaternary ammonium salts. Some POSS chemicals may be purchased commercially under the name Octaammonium POSS, by Hybrid Plastics of Hattiesburg, Miss. Conventional POSS materials have a molecular particle diameter ranging from about 7 nanometers to about 300 nanometers, which is typically much smaller than the diameter of colloidal silica particles and the like. Polymers containing POSS molecules exhibit extremely well dispersed POSS molecules at the nanometer scale, i. e. the polymers are true nanocomposites. The physical form of the material varies, and may be present in many forms ranging from liquid, to wax, to powder, to a crystalline solid. POSS structures can be functionalized with a wide variety of groups, leading to a range of POSS monomers. The nature of the functional group determines compatibility with the polymer matrix. A suitable POSS material includes Octaammonium POSS, comprising the structure of Formula 1:

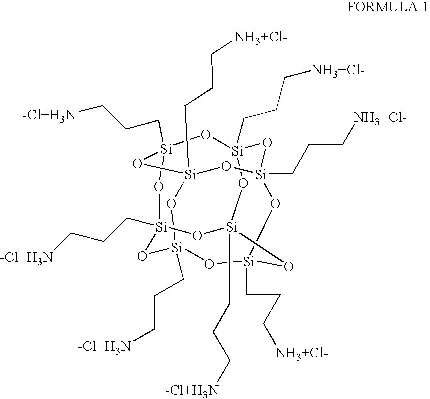

Regarding the non-leaching properties of the biocidal agent, and in particular the non-leaching properties of a biocidal agent as a component of a cellulosic composition (as described below), in certain embodiments it is preferred that from about 90% to about 100% of the biocidal agent present in the overall cellulosic composition is retained within the cellulosic composition or within an article formed by the cellulosic composition, during use applications in an outdoor environment.

The biocidal agent is preferably thermally stable at processing temperatures of the biocidal premixture, and retains its anti-biologic activity during use applications. It has been known to use moderately thermally stable anti-biologic additives in various polymeric applications. While not typically considered heat stable enough for use in polyolefins and other higher temperature processed polymers industry, these moderately thermally stable materials may retain their activity for use with cellulosic composite processing temperatures.

The biocidal agent may be combined with other components of the biocidal premixture in any suitable conventional manner such as mixing, blending, extrusion, pelletization, and the like. Most preferably, the biocidal agent is substantially uniformly dispersed throughout the overall biocidal premixture. In certain embodiments, the biocidal premixture may be present in the form of an emulsion. Such emulsions are particularly useful in biocidal coatings and the like.

Furthermore, it is preferred that, upon combining the biocidal premixture with a composite material, biocidal agent is preferably present such that it is available at the composite material's surfaces, including outer surfaces and/or internal pore surfaces of the composite material, in order to limit, inhibit, or prevent biological growth, formation, or spreading, on or in those surfaces of the composite material.

The biocidal agent is preferably present in an amount ranging from about 0.1 to about 50 percent by weight of the overall biocidal premixture, more preferably from about 1 to about 30 percent by weight of the biocidal premixture, and most preferably from about 1 to about 20 percent by weight of the biocidal premixture.

The biocidal agent may be present in the biocidal premixture in any suitable conventional form, nonexclusively including solids, liquids, waxes, powders, and the like. In one embodiment of the invention, a POSS biocidal agent is present in the biocidal premixture in the form of a powder, and in the range of from about 0.1 percent to about 10.0 percent by weight of the overall biocidal premixture.

In certain preferred embodiments of the invention, the biocidal premixture further comprises a coupling agent. The coupling agent serves to promote adhesion and/or enhance compatibility between the biocidal agent and components of a composite composition, such as the cellulose-containing composites described herein. A problem sometimes associated with cellulose-containing composites is the relative inability to bind cellulosic fibers and thermoplastic binder. In addition, adhesion instability between fillers and resinous mixtures may sometimes cause material failure. The presence of the coupling agent according to the present invention serves to improve adhesion stability in such composites. Inclusion of the coupling agent is further desirable in improving the flexural modulus of a shaped article containing the biocidal premixture of this invention.

In general, anhydride moieties or compounds with carboxylic functionality are suitable for use as a coupling agent in accordance with this invention. In one preferred embodiment, the coupling agent comprises a maleic anhydride functionality. Other preferred materials for the coupling agent nonexclusively include maleated polyolefins, maleated polyethylenes, maleated polypropylenes, and combinations thereof.

As used herein, the term “polyethylenes” refers to and includes homopolymers of polyethylene and all forms of polyethylene copolymers and terpolymers, including ethylene-propylene copolymers provided that at least the substantive majority of the polymer is formed of polyethylene moieties on a mole percent basis. Similarly, the term “polypropylenes” refers to and includes homopolymers of polypropylene and all forms of polypropylene copolymers and terpolymers, including propylene-ethylene copolymers, provided that at least the substantive majority of the polymer is formed of polypropylene moieties on a mole percent basis.

Examples of suitable maleated polyolefins include maleated derivatives of polyethylenes (including low density, high density, and linear low density polyethylenes), polypropylenes, polystyrenes and the like; co- and terpolymers including ethylene-butylene, EPDM (ethylene, propylene, diene monomer), EVA (ethylene-vinyl acetate), ethylene-butyl acrylate-carbon monoxide, and the like, and combinations thereof. Further examples of maleated polyolefins can be found in WO2005/021606, which is incorporated herein by reference.

The coupling agent is preferably present in the biocidal premixture in an amount ranging from about 1 to about 99 percent by weight of the overall premixture, more preferably from about 20 to about 80 percent by weight of the overall premixture, and most preferably from about 30 to about 70 percent by weight of the overall premixture. The coupling agent may be combined with other components of the biocidal premixture using any suitable conventional method such as mixing, blending, extrusion, pelletization, and the like.

In certain preferred embodiments of this invention, the biocidal composition comprises a lubricant. The lubricant serves to promote dispersion of the biocidal agent throughout the biocidal composition, and particularly throughout an overall composite composition such as the cellulosic compositions described below. The lubricant also serves to enhance polymeric properties and/or aid in processing, such as promoting extrudability and processability of the biocidal composition and of materials containing the biocidal composition. The term “extrudability” is generally understood in the art to signify the energy expended in extruding a workpiece. The term “processability” is generally understood to signify the energy expended in blending the composite constituents. The lubricant preferably comprises a material which reduces the interparticulate friction between components of the overall composite composition. This results in an improvement in the speed and/or efficiency with which a composite composition is effectively formed, and ultimately extruded into a shaped article. The lubricant may also serve to lower the loading requirement of materials, such as the biocidal agent, in an overall composite composition to achieve efficacy. Examples of suitable materials for the lubricant nonexclusively include polyol esters, alkyl esters, amide esters, fatty acid ester waxes, paraffin waxes, Fischer-Tropsch waxes, alpha olefins, microcrystalline waxes, amide waxes such as carboxyamide waxes, stearamide waxes, ethylene bis(stearamide) wax, polyethylene waxes including oxidized low and high density polyethylene waxes, and the like, and combinations thereof. Preferably, the lubricant comprises a material which is substantially free of metal stearates and metal carboxylates, which have been known to negatively affect the sustained performance of a processing operation when used as a lubricant. In certain embodiments of the invention, an overall composite composition comprising a biocidal premixture herein described, and having a lubricant present, small levels of zinc stearate may be tolerated, such as levels up to about 4% by weight of zinc stearate, preferably up to about 2% by weight of zinc stearate.

In one embodiment, the lubricant comprises a polyol ester formed by the reaction of polyol (polyhydroxyl compounds) with one or more mono- or poly-basic carboxylic acid or carboxylic acid functional groups. Suitable polyols nonexclusively include those represented by the general formula R(OH)ₙ where R is any aliphatic or cyclo-aliphatic hydrocarbyl group (preferably an alkyl) and n is an integer with a value of at least 2. The hydrocarbyl group may contain from about 2 to about 20 or more carbon atoms, and may also contain substituents such as chlorine, nitrogen and/or oxygen atoms. The polyhydroxyl compounds may generally contain one or more oxyalkylene groups such that the polyhydroxyl compounds include compounds such as polyetherpolyols. The number of carbon atoms and number of hydroxy groups in the polyhydroxyl compound used to form the carboxylic esters may vary.

In another embodiment of the present invention, alcohols are particularly useful as polyols selected from the group consisting of neopentyl glycol, trimethylolethane, trimethylolpropane, trimethylolbutane, mono-pentaerythritol, technical grade pentaerythritol, and di-pentaerythritol. In a further embodiment, the alcohols are of technical grade (e. g., approx. 88% mono-, 10% di- and 1-2% tri-pentaerythritol) pentaerythritol, monopentaerythritol, and di-pentaerythritol.

Carboxylic acids of the present invention preferably include any C₂ to C₂₀ mono- and di-acids, preferably adipic and stearic acid. Other effective coupling compounds may also be used. Functional derivatives of carboxylic acids may also be used to form the lubricant. For example, anhydrides of polybasic acids can be used in place of polybasic acids, when esters are being formed. Examples of such nonexclusively include succinic anhydride, glutaric anhydride, adipic anhydride, maleic anhydride, phthalic anhydride, trimellitic anhydride, nadic anhydride, methyl nadic anhydride, hexahydrophthalic anhydride, stearic anhydride and mixed anhydrides of polybasic acids, complex esters, and mixtures and combinations thereof.

In one embodiment of the present invention, a complex ester comprises a mixture of alkyl esters of Formula 2 below: 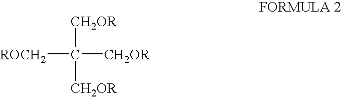
wherein
R is independently hydrogen, or —C(O)R′ and wherein at least one of R is —C(O)R′;
R′ is independently hydrogen, an unsaturated or saturated alkyl chain having from about 3 to about 18 carbon atoms, or —C(O)—X—COOH; and
X is an unsaturated or saturated alkyl chain that may be mono- or poly-valent, having from about 3 to about 18 carbon atoms.

In another embodiment of the invention, the alkyl ester comprises pentaerythritol adipate-stearate. The aforementioned moiety is a mixture of alkyl esters of Formula 2 wherein about 14% of the organic species are —C(O)—X—COOH moieties derived from adipic acid and about 71% of the organic species are —C(O)R′ moieties derived from stearic acid and its associated acids (typically, palmitic acid). Such is commercially available under the name RL 710 from Honeywell International, Inc. As previously stated, the lubricant of the present invention may include carboxyamide wax, stearamide wax, ethylene bis(stearamide) wax, and combinations thereof. A combination of alkyl ester and amide wax may be used over a wide range of relative concentrations within the lubricant. In one embodiment, the weight ratio of alkyl ester to amide wax may ranges from about from about 30:1 to about 1:1. In another embodiment, the weight ratio of alkyl ester to amide wax ranges about 20:1 to about 2:1. Other lubricants suitable for use in accordance with the present invention are commercially available from Honeywell International, Inc. under the trade name Rheochem. The lubricant is preferably present in an amount ranging from about 1 to about 99 percent by weight of the biocidal composition, more preferably from about 20 to about 80 percent by weight of the biocidal composition, and most preferably from about 30 to about 70 percent by weight of the biocidal composition

The lubricant may be combined with other components of the biocidal premixture using any conventional method such as heated blending and the like. The biocidal agent and/or coupling agent may or may not form a reaction product upon combining with the lubricant. In a preferred embodiment, the biocidal agent, coupling agent, and lubricant are present in the overall biocidal premixture in a ratio ranging from about 1:2:2 to about 2:1:1 respectively.

A further embodiment of the invention is directed to a biocidal compound comprising a biocidal agent which is nucleophilically grafted to the coupling agent. Nucleophilic grafting is done by reacting the components to form a covalent bond between the biocidal agent and the coupling agent. The nucleophilic grafting serves to bind the biocidal agent to the coupling agent, to thereby prevent or inhibit leaching of the biocide. In such an embodiment wherein the biocidal agent and coupling agent are nucleophilically grafted, the biocidal agent preferably comprises a primary amine group capable of nucleophilic substitution reaction with the coupling agent. Examples of suitable biocidal agents having such primary amine groups nonexclusively include sulfadiazine, diiodomethyl-p-tolylsulfone, an ammonium salt, and combinations thereof. Suitable coupling agents nonexclusively include those described above.

An additional embodiment of the invention is directed to a biocidal premixture comprising a biocidal compound, as described herein, and a lubricant. Suitable lubricants nonexclusively include those described above.

Any of the biocidal premixtures described above can be used in the formation of composite compositions for use in various manufacturing applications and technology areas. The inventive biocidal premixtures are particularly useful in the formation of composite cellulosic compositions. Such cellulosic compositions comprise a cellulosic material, a thermoplastic polymer, and a biocidal premixture of the present invention. A preferred process for forming such a cellulosic compositions includes first forming a biocidal premixture as described above, and thereafter combining the biocidal premixture with a cellulosic material and a thermoplastic polymer.

The cellulosic material serves as a structural component of the overall cellulosic composition, and of shaped articles formed therefrom. The cellulosic material may include any cellulose-based material, nonexclusively including various types of wood and wood products such as wood flour, wood pulp or wood fibers; paper; tree bark; straw; hay; cotton; hemp; flax; plants and plant components such as leaves, fruits, seeds, pits, flowers, nut shells and the like; grains; rice hull; corn silk; corn husks; and the like, and combinations thereof. In a preferred embodiment, the cellulosic material comprises wood. The cellulosic material may be present in any suitable form such as particles, fibers, flakes, pulp, chips, paper, shavings, sawdust, flours, cellulose-containing byproducts and the like, and combinations thereof. The cellulosic material is preferably present in an amount ranging from about 40 to 90 percent by weight of the cellulosic composition, more preferably from about 50 to about 80 percent by weight of the cellulosic composition, and most preferably from about 55 to about 65 percent by weight of the cellulosic composition.

The thermoplastic polymer serves to promote the combining of a cellulosic material with other components of the cellulosic composition, via melt blending or other conventional methods, and allow effective formation of the cellulosic composition into shaped articles using extrusion, molding or other conventional methods. The thermoplastic polymer may comprise any suitable conventionally known thermoplastic polymer. Some examples of suitable thermoplastic polymers nonexclusively include materials selected from the group consisting of polyamides, polyesters, polyolefins, polyphenylene sulfides, polyoxymethylenes, styrene polymers, polycarbonates, and combinations thereof. As used in this invention, the term polyolefin refers to homopolymers, copolymers and modified polymers of unsaturated aliphatic hydrocarbons. In some embodiments, polyethylene and polypropylene may be used. In other embodiments, high-density polyethylene (HDPE) may be used. The thermoplastic polymer may be used in virgin form as well as recycled (waste) form.

The thermoplastic polymer is preferably present in an amount ranging from about 10 to 40 percent by weight of the cellulosic composition, more preferably from about 20 to about 40 percent by weight of the cellulosic composition, and most preferably from about 25 to about 35 percent by weight of the cellulosic composition.

The biocidal premixture is preferably present in the cellulosic composition an amount ranging from about 0.1 to 50 percent by weight of the overall cellulosic composition, more preferably from about 1 to about 40 percent by weight of the cellulosic composition, and most preferably from about 1 to about 30 percent by weight of the cellulosic composition. In one preferred embodiment, the biocidal premixture is present in the cellulosic composition in an amount such that the biocidal agent is present in an amount ranging from about 0.5 about 10 percent by weight of the overall cellulosic composition, about the coupling agent is present in an amount ranging from about 1 to about 5 percent by weight of the overall cellulosic composition, about the lubricant is present in an amount ranging from about 1 to about 5 percent by weight of the overall cellulosic composition.

The cellulosic composition may further comprise other materials such as organic or inorganic fillers, reinforcements, and the like. Examples of suitable fillers and reinforcement materials nonexclusively include inorganic fillers such as glass fiber, carbon fiber, talc, mica, kaolin, calcium carbonate and the like. Organic fillers nonexclusively include cellulosic materials, polymeric fibers, and the like. Such may be present in amounts easily determined by those skilled in the art. Suitable amounts range from about 1 to about 10 percent by weight of the overall cellulosic composition.

The cellulosic compositions of the invention may be formed using any suitable conventional methods such as mixing, blending, extrusion, pelletization, and the like. In one preferred embodiment, components of the cellulosic composition are combined in a mixer and blended until a homogeneous mix is achieved. The cellulosic compositions are preferably further formed into shaped articles such as wood-plastic composite articles. Formation of shaped articles may be done using conventional methods such as extruding, injection molding, and the like. Such shaped articles particularly useful for exterior applications, nonexclusively including decks, siding, roofing, windows, moldings, docks, wharfs and the like.

Shaped articles comprising the inventive biocidal premixtures offer a significant improvement in performance over conventionally known materials. Compositions that include a maleic anhydride-based coupling agent and a lubricant produce shaped articles with flexural modulus properties of at least about 25 relative percent, and often up to 75 relative percent, greater than similar compositions containing conventionally used lubricants. In one preferred embodiment, articles containing the biocidal premixture of the present invention provide exhibit a flexural modulus ranging from about 1000 psi to about 5000 psi. Preferably such articles have a flexural modulus of from about 2000 psi, more preferably about 2500 psi or more, and most preferably about 3000 psi or more.

Materials containing the biocidal premixtures of this invention exhibit improved resistance to attack by molds, fungus, algae, and the like, improved adhesion stability, greater moisture resistance and aging resistance, limited leaching, enhanced toughness moduli, and increased efficacy.

The following non-limiting examples serve to illustrate the invention. It will be appreciated that variations in proportions and alternatives in elements of the components of the invention will be apparent to those skilled in the art and are within the scope of the present invention.

### EXAMPLE 1

A biocidal premixture is prepared by mixing an Octaammonium POSS biocidal agent in an amount of 5% by weight of the overall biocidal premixture with a maleic anhydride coupling agent in an amount of 95% by weight of the overall biocidal premixture. A cellulosic composition is then prepared by mixing the biocidal premixture in an amount of 5% by weight of the overall cellulosic composition with wood fiber in an amount of 65% by weight of the overall cellulosic composition and an HDPE thermoplastic polymer in an amount of 30% by weight of the overall cellulosic composition. The cellulosic composition is then extruded to form a composite shaped article.

### EXAMPLE 2

A biocidal premixture is prepared by mixing an Octaammonium POSS biocidal agent in an amount of 5% by weight of the overall biocidal premixture with a carboxyamide wax lubricant in an amount of 95% by weight of the overall biocidal premixture. A cellulosic composition is then prepared by mixing the biocidal premixture in an amount of 5% by weight of the overall cellulosic composition with wood fiber in an amount of 65% by weight of the overall cellulosic composition and an HDPE thermoplastic polymer in an amount of 30% by weight of the overall cellulosic composition. The cellulosic composition is then extruded to form a composite shaped article.

### EXAMPLE 3

A biocidal premixture is prepared by mixing an Octaammonium POSS biocidal agent in an amount of 5% by weight of the overall biocidal premixture with a maleic anhydride coupling agent in an amount of 40% by weight of the overall biocidal premixture and a carboxyamide wax lubricant in an amount of 55% by weight of the overall biocidal premixture. A cellulosic composition is then prepared by mixing the biocidal premixture in an amount of 5% by weight of the overall cellulosic composition with wood fiber in an amount of 65% by weight of the overall cellulosic composition and an HDPE thermoplastic polymer in an amount of 30% by weight of the overall cellulosic composition. The cellulosic composition is then extruded to form a composite shaped article.

### EXAMPLE 4

A biocidal premixture is prepared by mixing a carboxyamide wax lubricant in an amount of 95% by weight of the overall biocidal premixture, with a biocidal compound in an amount of 5% by weight of the overall biocidal premixture, which biocidal compound includes a sulfadiazine biocidal agent nucleophilically grafted to a maleic anhydride coupling agent. A cellulosic composition is then prepared by mixing the biocidal premixture in an amount of 5% by weight of the overall cellulosic composition with wood fiber in an amount of 65% by weight of the overall cellulosic composition and an HDPE thermoplastic polymer in an amount of 30% by weight of the overall cellulosic composition. The cellulosic composition is then extruded to form a composite shaped article.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A biocidal premixture comprising a combination of a biocidal agent and at least one or both of:
a) a coupling agent; and/or
b) a lubricant.

2. The biocidal premixture of claim 1 which comprises a coupling agent but not a lubricant.

3. The biocidal premixture of claim 1 which comprises a lubricant but not a coupling agent.

4. The biocidal premixture of claim 1 which comprises both a coupling agent and a lubricant.

5. The biocidal premixture of claim 1, wherein the biocidal agent comprises a material selected from the group consisting of algicides, herbicides, antifungals, antimicrobials, antibacterials, antibiotics, termiticides, insecticides, miticides, and combinations thereof.

6. The biocidal premixture of claim 1, wherein the biocidal agent comprises a material of the formula:

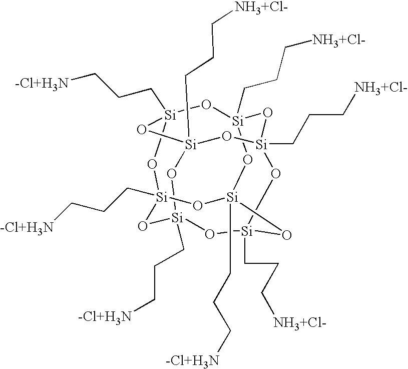

7. The biocidal premixture of claim 1, wherein the coupling agent comprises a maleated polyolefin, a maleated polyethylene, a maleated polypropylene, or combinations thereof.

8. The biocidal premixture of claim 1, wherein the coupling agent comprises maleic anhydride.

9. The biocidal premixture of claim 1, wherein the lubricant comprises a material which is substantially free of metal stearates and metal carboxylates.

10. The biocidal premixture of claim 1, wherein the lubricant comprises a polyol ester or an alkyl ester of a carboxylic acid.

11. The biocidal premixture of claim 1, wherein the lubricant comprises a carboxyamide wax, a stearamide wax or combinations thereof.

12. The biocidal premixture of claim 1, wherein the biocidal agent is present in an amount ranging from about 1 to about 30 parts by weight of the premixture.

13. The biocidal premixture of claim 1, wherein the coupling agent is present in an amount ranging from about 1 to about 70 parts by weight of the premixture.

14. The biocidal premixture of claim 1, wherein the lubricant is present in an amount ranging from about 1 to about 70 parts by weight of the premixture.

15. A cellulosic composition which comprises:
a) a cellulosic material;
b) a thermoplastic polymer; and
c) the biocidal premixture comprising a combination of a biocidal agent and at least one or both of:
i) a coupling agent; and/or
ii) a lubricant.

16. The cellulosic composition of claim 15 wherein the cellulosic material comprises wood.

17. The cellulosic composition of claim 15 wherein the thermoplastic polymer comprises a material selected from the group consisting of polyamides, polyesters, polyolefins, polyphenylene sulfides, polyoxymethylenes, styrene polymers, polycarbonates, and combinations thereof.

18. A shaped article comprising the biocidal premixture of claim 1.

19. A process for forming a cellulosic composition which comprises:
a) first forming a biocidal premixture comprising a combination of a biocidal agent and at least one or both of:
i) a coupling agent; and/or
ii) a lubricant; and
b) thereafter combining the biocidal premixture with a cellulosic material and a thermoplastic polymer to thereby form a cellulosic composition.

20. A shaped article comprising a cellulosic composition formed by the process of claim 19.

21. A biocidal compound comprising a biocidal agent nucleophilically grafted to a coupling agent.

22. The biocidal compound of claim 21, wherein the biocidal agent comprises a primary amine group capable of nucleophilic substitution reaction with the coupling agent.

23. The biocidal compound of claim 21 wherein the biocidal agent is selected from the group consisting of sulfadiazine, diiodomethyl-p-tolylsulfone, an ammonium salt, and combinations thereof.

24. The biocidal compound of claim 21, wherein the coupling agent comprises a maleated polyolefin, a maleated polyethylene, a maleated polypropylene, or combinations thereof.

25. A biocidal premixture comprising the biocidal compound of claim 21 and a lubricant.
